(19) United States
(12) Patent Application Publication
(10) Pub. No.: US 2023/0065820 A1
(43) Pub. Date: Mar. 2, 2023

(54) TELEHEALTH AND MICRODOSING PLATFORM FOR PSYCHEDELIC-ASSISTED THERAPY

(71) Applicant: PSILOTHERA, INC., Henderson, NV (US)

(72) Inventors: Charles John Atanasio, JR., Henderson, NV (US); John Patrick Atanasio, SR., Henderson, NV (US)

(21) Appl. No.: 17/459,998

(22) Filed: Aug. 27, 2021

(51) Int. Cl. G16H 50/20; G16H 10/20; G16H 15/00; G16H 80/00; G16H 20/70

(52) U.S. Cl. CPC G16H 50/20; G16H 10/20; G16H 15/00; G16H 80/00; G16H 20/70

(57) ABSTRACT

One embodiment provides a method comprising providing to a patient user a questionnaire comprising questions inquiring about well-being of the patient user, receiving responses of the patient user to the questionnaire, and generating a patient user profile by applying machine learning to the responses. The patient user profile comprises diagnostic questions customized for the patient user. The method further comprises providing the patient user profile to a third party user, and initiating a video teleconference between the patient user and the third party user during which the third party user asks the patient user the diagnostic questions to assess and diagnose a mental health of the patient user. The method further comprises providing the patient user with a therapeutic treatment plan prescribed by the third party user based on the assessment and diagnosis. The plan indicates at least one dosing session involving psychedelics for the patient user to complete.

### TECHNICAL FIELD

One or more embodiments relate generally to telehealth, and in particular, a telehealth platform for psychedelic-assisted therapy.

### BACKGROUND

Telehealth is the distribution of health-related services and information via electronic information and telecommunication technologies. It allows long-distance patient and clinician contact, care, advice, reminders, education, intervention, monitoring, and remote admissions.

Psychedelic therapy is a type of psychiatric practice that involves ingesting a psychedelic substance as part of a psychotherapeutic process. In psychedelic therapy, the use of psychedelics is typically combined with talk therapy. Recent studies have shown that psychedelic treatment with psilocybin relieves major depression.

### SUMMARY

One embodiment provides a method comprising providing to a patient user a questionnaire comprising questions inquiring about well-being of the patient user, receiving responses of the patient user to the questionnaire, and generating a patient user profile by applying machine learning to the responses. The patient user profile comprises diagnostic questions customized for the patient user. The method further comprises providing the patient user profile to a third party user, and initiating a video teleconference between the patient user and the third party user. The third party user asks the patient user the diagnostic questions during the video teleconference to assess and diagnose the mental health of the patient user. The method further comprises providing the patient user with a therapeutic treatment plan prescribed by the third party user based on the assessment and diagnosis. The therapeutic treatment plan is indicative of at least one dosing session for the patient user to complete, frequency of the at least one dosing session, and an amount of psychedelics for the patient user to consume during the at least one dosing session.

Another embodiment provides a system comprising at least one processor and a non-transitory processor-readable memory device storing instructions that when executed by the at least one processor causes the at least one processor to perform operations. The operations include providing to a patient user a questionnaire comprising questions inquiring about well-being of the patient user, receiving responses of the patient user to the questionnaire, and generating a patient user profile by applying machine learning to the responses. The patient user profile comprises diagnostic questions customized for the patient user. The operations further comprise providing the patient user profile to a third party user, and initiating a video teleconference between the patient user and the third party user. The third party user asks the patient user the diagnostic questions during the video teleconference to assess and diagnose the mental health of the patient user. The operations further comprise providing the patient user with a therapeutic treatment plan prescribed by the third party user based on the assessment and diagnosis. The therapeutic treatment plan is indicative of at least one dosing session for the patient user to complete, frequency of the at least one dosing session, and an amount of psychedelics for the patient user to consume during the at least one dosing session.

One embodiment provides a non-transitory processor-readable medium that includes a program that when executed by a processor performs a method comprising providing to a patient user a questionnaire comprising questions inquiring about well-being of the patient user, receiving responses of the patient user to the questionnaire, and generating a patient user profile by applying machine learning to the responses. The patient user profile comprises diagnostic questions customized for the patient user. The method further comprises providing the patient user profile to a third party user, and initiating a video teleconference between the patient user and the third party user. The third party user asks the patient user the diagnostic questions during the video teleconference to assess and diagnose the mental health of the patient user. The method further comprises providing the patient user with a therapeutic treatment plan prescribed by the third party user based on the assessment and diagnosis. The therapeutic treatment plan is indicative of at least one dosing session for the patient user to complete, frequency of the at least one dosing session, and an amount of psychedelics for the patient user to consume during the at least one dosing session.

These and other features, aspects and advantages of the one or more embodiments will become understood with reference to the following description, appended claims and accompanying figures.

### BRIEF DESCRIPTION OF THE DRAWINGS

FIG. 1 illustrates an example computing architecture for implementing a telehealth platform for psychedelic-assisted therapy, in one or more embodiments;

FIG. 2 illustrates one or more example components of the telehealth system utilized in the training phase, in one or more embodiments;

FIG. 3 illustrates one or more example components of the telehealth system utilized in the deployment phase, in one or more embodiments;

FIG. 4 illustrates one or more example components of the patient enrollment system utilized in the deployment phase, in one or more embodiments;

FIG. 5 illustrates one or more example components of the preparation system utilized in the deployment phase, in one or more embodiments;

FIG. 6 illustrates one or more example components of the administration system utilized in the deployment phase, in one or more embodiments;

FIG. 7 illustrates one or more example components of the integration system utilized in the deployment phase, in one or more embodiments;

FIG. 8 is a flowchart of an example process for implementing a telehealth platform for psychedelic-assisted therapy, in one or more embodiments; and

FIG. 9 is a high-level block diagram showing an information processing system comprising a computer system useful for implementing the disclosed embodiments.

### DETAILED DESCRIPTION

The following description is made for the purpose of illustrating the general principles of one or more embodiments and is not meant to limit the inventive concepts claimed herein. Further, particular features described herein can be used in combination with other described features in each of the various possible combinations and permutations. Unless otherwise specifically defined herein, all terms are to be given their broadest possible interpretation including meanings implied from the specification as well as meanings understood by those skilled in the art and/or as defined in dictionaries, treatises, etc.

One or more embodiments relate generally to telehealth, and in particular, a telehealth platform for psychedelic-assisted therapy. One embodiment provides a method comprising providing to a patient user a questionnaire comprising questions inquiring about well-being of the patient user, receiving responses of the patient user to the questionnaire, and generating a patient user profile by applying machine learning to the responses. The patient user profile comprises diagnostic questions customized for the patient user. The method further comprises providing the patient user profile to a third party user, and initiating a video teleconference between the patient user and the third party user. The third party user asks the patient user the diagnostic questions during the video teleconference to assess and diagnose a mental health of the patient user. The method further comprises providing the patient user with a therapeutic treatment plan prescribed by the third party user based on the assessment and diagnosis. The therapeutic treatment plan is indicative of at least one dosing session for the patient user to complete, frequency of the at least one dosing session, and an amount of psychedelics for the patient user to consume during the at least one dosing session.

Another embodiment provides a system comprising at least one processor and a non-transitory processor-readable memory device storing instructions that when executed by the at least one processor causes the at least one processor to perform operations. The operations include providing to a patient user a questionnaire comprising questions inquiring about well-being of the patient user, receiving responses of the patient user to the questionnaire, and generating a patient user profile by applying machine learning to the responses. The patient user profile comprises diagnostic questions customized for the patient user. The operations further comprise providing the patient user profile to a third party user, and initiating a video teleconference between the patient user and the third party user. The third party user asks the patient user the diagnostic questions during the video teleconference to assess and diagnose the mental health of the patient user. The operations further comprise providing the patient user with a therapeutic treatment plan prescribed by the third party user based on the assessment and diagnosis. The therapeutic treatment plan is indicative of at least one dosing session for the patient user to complete, frequency of the at least one dosing session, and an amount of psychedelics for the patient user to consume during the at least one dosing session.

One embodiment provides a non-transitory processor-readable medium that includes a program that when executed by a processor performs a method comprising providing to a patient user a questionnaire comprising questions inquiring about well-being of the patient user, receiving responses of the patient user to the questionnaire, and generating a patient user profile by applying machine learning to the responses. The patient user profile comprises diagnostic questions customized for the patient user. The method further comprises providing the patient user profile to a third party user, and initiating a video teleconference between the patient user and the third party user. The third party user asks the patient user the diagnostic questions during the video teleconference to assess and diagnose the mental health of the patient user. The method further comprises providing the patient user with a therapeutic treatment plan prescribed by the third party user based on the assessment and diagnosis. The therapeutic treatment plan is indicative of at least one dosing session for the patient user to complete, frequency of the at least one dosing session, and an amount of psychedelics for the patient user to consume during the at least one dosing session.

For expository purposes, the term “patient user” as used in this specification generally refers to an individual seeking mental health treatment for a disorder such as, but not limited to, depression, anxiety, post-traumatic stress disorder (PTSD), attention-deficit/hyperactivity disorder (ADHD), an eating disorder, an addiction (e.g., opioid addiction), Alzheimer's, dementia, etc.

For expository purposes, the term “third party user” as used in this specification generally refers to an individual or an entity providing healthcare such as, but not limited to, a clinician, a psychiatrist, a doctor, a physician, a practitioner, a mental health therapist, a mental health clinic or facility, a healthcare worker, a healthcare provider, a licensed mental health professional, an individual licensed for psilocybin therapy, etc.

For expository purposes, the term “therapeutic treatment plan” as used in this specification generally refers to a plan/program to meet a desired mental health goal (e.g., decrease anxiety) of a patient user using psychedelics, such as psilocybin. A therapeutic treatment plan outlines a schedule of one or more dosing sessions a patient user should participate in over a pre-determined period of time to improve their mental health and meet a desired mental health goal. A dosing session is either a microdosing session or a high dose psychedelic-assisted therapy session (“high dose session”). A therapeutic treatment plan identifies intensity of dosing sessions (e.g., microdosing sessions and/or high dose sessions) and frequency of the dosing sessions (e.g., when each microdosing session and/or high dose session should occur) over a pre-determined duration/period of time (e.g., one year).

For psilocybin therapy, a microdosing session involves a patient user taking/ingesting a prescribed low dose of psilocybin. During a microdosing session, supervision of a patient user by a third party user is not necessary. A high dose session involves a patient user taking/ingesting a prescribed high dose of psilocybin under the guidance, supervision, and observation of a third party user (e.g., physician-directed therapy).

Each patient user is assigned at least one third party user tasked to make an assessment of mental health (“mental health assessment”) and a mental health diagnosis of the patient user, prescribe a therapeutic treatment plan to the patient user, guide, supervise, and observe the patient user during a high dose session, and/or monitor experience/satisfaction of the patient user with the therapeutic treatment plan.

Telehealth is an increasingly valuable method for improving mental and physical wellness of users, lowering cost of individualized care, and increasing efficacy of treatments. A third party user may assign one or more mobile/wearable devices (e.g. a smartphone, a smartwatch, etc.) or Internet-of-Things (IoT) devices (e.g., an environmental IoT sensor, etc.) to a patient user as part of a complete therapeutic treatment plan to track one or more vitals of the patient user. Each assigned device collects user data from one or more sensors units that capture metrics associated with various attributes of the patient user's experience with microdosing, such as anxiety/stress levels, amount of sleep, etc. The collected user data may be sent to an online service for review by a third party user assigned to the patient user to determine whether the patient user is satisfied with the therapeutic treatment plan and making progress toward a desired mental health goal (i.e., tele-mental healthcare).

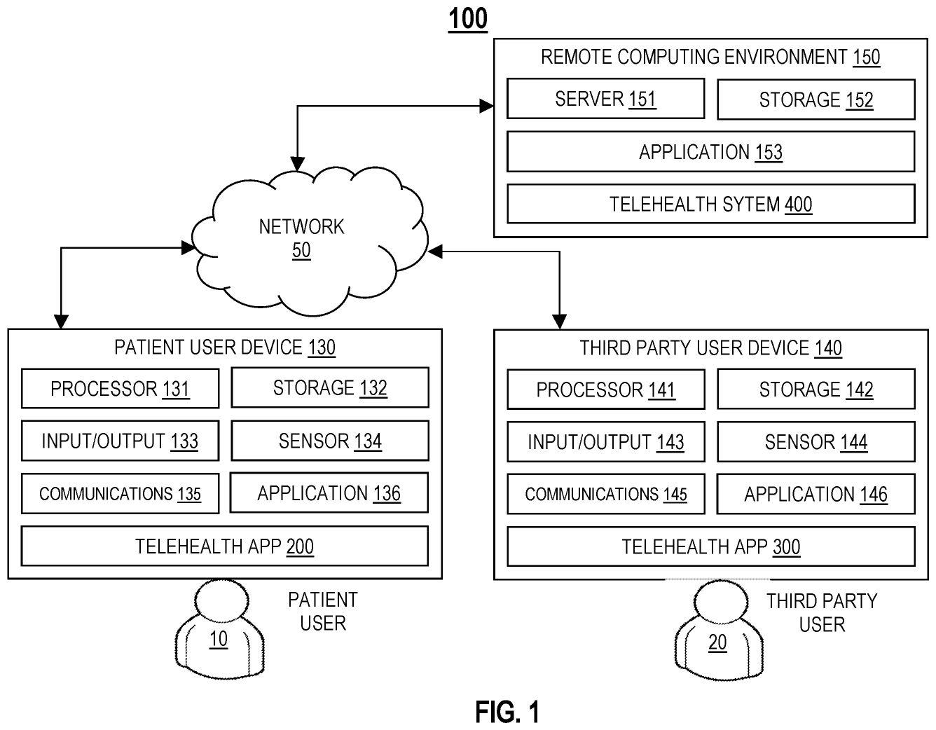

FIG. 1 illustrates an example computing architecture 100 for implementing a telehealth platform for psychedelic-assisted therapy, in one or more embodiments. The computing architecture 100 comprises a remote computing environment 150. The remote computing environment 150 includes resources such as, but not limited to, one or more server devices 151 and one or more storage devices 152. One or more applications 153 that provide higher-level services may execute/operate on the remote computing environment 150 utilizing the resources of the remote computing environment 150.

In one embodiment, the applications 153 include one or more components of a telehealth system 400 for psychedelic-assisted therapy. As described in detail later herein, the telehealth system 400 facilitates psilocybin therapy between one or more patient users 10 and one or more third party users 20, such as assessing and diagnosing mental health of a patient user 10, prescribing a therapeutic treatment plan to the patient user 10 based on the assessment and diagnosis, and determining experience/satisfaction of the patient user 10 with the therapeutic treatment plan. The telehealth system 400 provides tele-mental healthcare.

The telehealth system 400 is configured to exchange data with one or more devices over a communications network/connection 50 (e.g., a wireless connection such as a Wi-Fi connection or a cellular data connection, a wired connection, or a combination of the two), such as one or more patient user devices 130 and one or more third party user devices 140.

In one embodiment, the computing architecture 100 comprises: at least one patient user device 130 that at least one patient user 10 has access to for interaction (e.g., a patient user device 130 positioned within proximity of a patient user 10), and at least one third party user device 140 that at least one third party user 20 has access to for interaction (e.g., a third party user device 140 positioned within proximity of a third party user 20). Examples of a patient user device 130 or a third party user device 140 include, but are not limited to, a mobile electronic device (e.g., a smart phone, a laptop, etc.), a wearable device (e.g., a smart watch), a video camera, an Internet of things (IoT) device, etc.

In one embodiment, a patient user device 130 includes resources, such as one or more processor units 131 and one or more storage units 132. One or more applications 136 may execute/operate on the patient user device 130 utilizing the resources of the patient user device 130. In one embodiment, the one or more applications 136 on the patient user device 130 include one or more software mobile applications loaded onto or downloaded to the patient user device 130.

In one embodiment, the one or more software mobile applications include a telehealth application (“app”) 200 configured to exchange data with the telehealth system 400 and at least one third party user device 140. As described in detail later herein, the telehealth app 200 is configured to facilitate one or more telehealth sessions involving a patient user 10 positioned within proximity of the patient user device 130. The telehealth app 200 is further configured to provide communications (e.g., emails, alerts/notifications, etc.) to the patient user 10 regarding a prescribed therapeutic treatment plan, and monitor experience/satisfaction of the patient user 10 with the therapeutic treatment plan. The telehealth app 200 provides the patient user 10 with a portal to the telehealth system 400.

In one embodiment, the one or more software mobile applications include other apps, such as a camera app, a social media app, a video streaming app, a messaging app, a video teleconferencing app (e.g., Microsoft Teams®, Zoom®), etc. In one embodiment, the telehealth app 400 is configured to invoke another app on the patient user device 130. For example, the telehealth app 400 invokes a video teleconferencing app to initiate a video teleconferencing session between the patient user 10 and a third-party user 20.

In one embodiment, the patient user device 130 comprises one or more sensor units 134 integrated in or coupled to the patient user device 130, such as a camera, a microphone, a GPS, a motion sensor, an accelerometer, a heart-rate sensor, a blood pressure sensor, etc.

In one embodiment, the patient user device 130 comprises one or more I/O units 133 integrated in or coupled to the patient user device 130. In one embodiment, the one or more I/O units 133 include, but are not limited to, a physical user interface (PUI) and/or a GUI, such as a keyboard, a keypad, a touch interface, a touch screen, a knob, a button, a display screen, etc. In one embodiment, the patient user 10 can utilize at least one I/O unit 133 to configure one or more user preferences, configure one or more parameters, provide user input (e.g., responses to a questionnaire), etc.

As described in detail later herein, data captured by the patient user device 130 (via the one or more I/O units 133 and/or the one or more sensor units 134) is forwarded to the telehealth system 400 for determining experience/satisfaction of the patient user 10 with the therapeutic treatment plan.

In one embodiment, the patient user device 130 comprises a communications unit 135 configured to exchange data with the telehealth system 400 and/or a third party user device 140 over the communications network/connection 50 (e.g., a wireless connection such as a Wi-Fi connection or a cellular data connection, a wired connection, or a combination of the two). The communications unit 135 may comprise any suitable communications circuitry operative to connect to a communications network and to exchange communications operations and media between the patient user device 130 and other devices connected to the same communications network 50. The communications unit 135 may be operative to interface with a communications network using any suitable communications protocol such as, for example, Wi-Fi (e.g., an IEEE 802.11 protocol), Bluetooth high frequency systems (e.g., 900 MHz, 2.4 GHz, and 5.6 GHz communication systems), infrared, GSM, GSM plus EDGE, CDMA, quadband, and other cellular protocols, VOIP, TCP-IP, or any other suitable protocol.

To facilitate tele-mental health (i.e., tele-mental healthcare), a third party user 20 assigned to the patient user 10 may utilize remote user monitoring to track progress of the patient user 10 towards achieving a desired mental health goal of the therapeutic treatment plan.

To facilitate remote user monitoring, the patient user 10 may utilize one or more patient user devices 130 to track/collect data relating to the patient user 10. Some of the data tracked/collected may be done in a real-time manner. If the patient user 10 utilizes multiple patient user devices 130, the patient user devices 130 may work separately or in tandem. Data tracked/collected by one patient user device 130 (e.g., a smartwatch) may be synced to another patient user device 130 (e.g., a smartphone) at a later time. For example, a smartphone carried by the patient user 10 may be paired with one or more other patient user devices 130 located in a car, home, and/or office of the patient user 10. The telehealth system 400 analyzes data tracked/collected by an individual patient user device 130, or through paired patient user devices 130, to determine experience/satisfaction of the patient user 10 with the therapeutic treatment plan.

In one embodiment, a third party user device 140 includes resources, such as one or more processor units 141 and one or more storage units 142. One or more applications 146 may execute/operate on the third party user device 140 utilizing the resources of the third party user device 140. In one embodiment, the one or more applications 146 on the third party user device 140 include one or more software mobile applications loaded onto or downloaded to the third party user device 140.

In one embodiment, the one or more software mobile applications include a telehealth app 300 configured to exchange data with the telehealth system 400 and at least one patient user device 130. As described in detail later herein, the telehealth app 300 is configured to facilitate one or more telehealth sessions involving a third party user 20 positioned within proximity of the third party user device 140. The telehealth app 300 is further configured to provide communications (e.g., emails, alerts/notifications, etc.) to the third party user 20 regarding information pertaining to a patient user 10 that the third party user 20 is assigned to. For example, in one embodiment, the telehealth app 300 may send an alert to notify the third party member 20 of a satisfaction of the patient user 10 with the therapeutic treatment plan. The alert may prompt/invoke the third party member 20 to take action, such as directly contacting the patient user 10 or adjusting the therapeutic treatment plan to improve their satisfaction. The telehealth app 300 provides the third party user 20 with a portal to the telehealth system 400.

In one embodiment, the one or more software mobile applications include other apps, such as a camera app, a social media app, a video streaming app, a messaging app, a video teleconferencing app (e.g., Microsoft Teams®, Zoom®), etc. In one embodiment, the telehealth app 400 is configured to invoke another app on the third party user device 140. For example, the telehealth app 400 invokes a video teleconferencing app to initiate a video teleconferencing session between the third-party user 20 and a patient user 10.

In one embodiment, the third party user device 140 comprises one or more sensor units 144 integrated in or coupled to the third party user device 140, such as a camera, a microphone, etc.

In one embodiment, the third party user device 140 comprises one or more I/O units 143 integrated in or coupled to the third party user device 140. In one embodiment, the one or more I/O units 143 include, but are not limited to, a physical user interface (PUI) and/or a GUI, such as a keyboard, a keypad, a touch interface, a touch screen, a knob, a button, a display screen, etc. In one embodiment, the third party user 20 can utilize at least one I/O unit 143 to configure one or more user preferences, configure one or more parameters, provide user input (e.g., responses relating to a mental status examination of a patient user 10), etc.

In one embodiment, the third party user device 140 comprises a communications unit 145 configured to exchange data with the telehealth system 400 and/or a patient user device 130 over the communications network/connection 50 (e.g., a wireless connection such as a Wi-Fi connection or a cellular data connection, a wired connection, or a combination of the two). The communications unit 145 may comprise any suitable communications circuitry operative to connect to a communications network and to exchange communications operations and media between the third party user device 140 and other devices connected to the same communications network 50. The communications unit 145 may be operative to interface with a communications network using any suitable communications protocol such as, for example, Wi-Fi (e.g., an IEEE 802.11 protocol), Bluetooth®, high frequency systems (e.g., 900 MHz, 2.4 GHz, and 5.6 GHz communication systems), infrared, GSM, GSM plus EDGE, CDMA, quadband, and other cellular protocols, VOIP, TCP-IP, or any other suitable protocol.

In one embodiment, a remote computing environment 150 provides an online platform for hosting one or more online services and/or distributing one or more software mobile applications. For example, a telehealth app 200, 300 may be loaded onto or downloaded to a patient user device 130, a third party user device 140 from a remote computing environment 150 that maintains and distributes updates for the telehealth app 200, 300. As another example, a remote computing environment 150 may comprise an online secure cloud computing environment providing shared pools of configurable computing system resources and higher-level services.

In one embodiment, operation of the telehealth system 400 may be divided into two different operating phases—a training phase and a deployment phase.

In one embodiment, in the training phase, the telehealth system 400 is configured to: (1) collect training data from one or more patient users 10 participating in one or more therapeutic treatment plans, and (2) based on the collected training data, train, using artificial intelligence (AI), a plurality machine learning models for use in the deployment phase.

In one embodiment, in the deployment phase, the telehealth system 400 is configured to perform: (1) facilitate a telehealth session between a patient user 10 and at least one third party user 20, (2) provide the patient user 10 with a customized therapeutic treatment plan prescribed by the at least one third party user 20, (3) collect user data from the patient user 10 to determine experience/satisfaction of the patient user 10 with the therapeutic treatment plan, and (4) provide communications to the patient user 10 and at least one third party user 20 assigned to the patient user 10 during the duration of the therapeutic treatment plan. In one embodiment, communications to the patient user 10 include, but are not limited to, emails and/or alerts/notifications of scheduled/upcoming dosing sessions for the patient user 10, requests for the patient user 10 to provide feedback describing their experience/satisfaction with a completed dosing session, etc. In one embodiment, communications to a third party user 20 include, but are not limited to, emails and/or alerts/notifications of scheduled/upcoming high dose sessions the third party user 20 will guide, supervise, and observe, requests and/or recommendations to adjust a prescribed therapeutic treatment plan, etc.

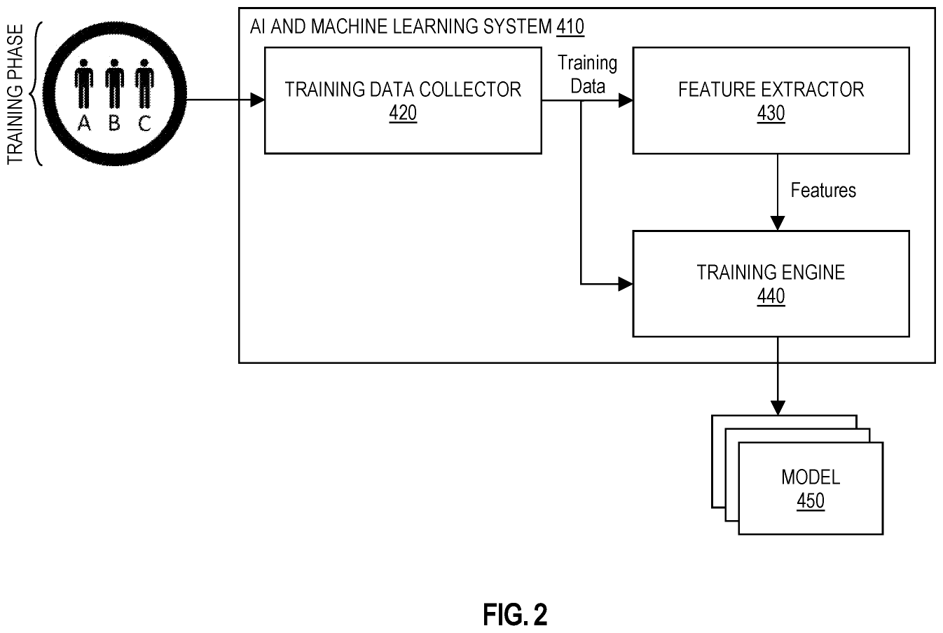

FIG. 2 illustrates one or more example components of the telehealth system 400 utilized in the training phase, in one or more embodiments. In one embodiment, the telehealth system 400 comprises an AI and machine learning system 410 utilized in the training phase.

In one embodiment, the AI and machine learning system 410 comprises a training data collector 420 configured to collect training data from one or more patient users 10 (e.g., user A, user B, and user C) and/or one or more third party users 20 assigned to the one or more patient users 10. For each of the one or more patient users 10, the training data is indicative of an experience/satisfaction of the patient user 10 with a prescribed therapeutic treatment plan.

In one embodiment, for each of the one or more patient users 10 (e.g., user A, user B, and user C), the training data comprises data tracked/collected by one or more patient user devices 130 associated with the patient user 10 such as, but not limited to, sensor data for the patient user 10 captured via one or more sensor units 134 (e.g., vitals of the patient user 10), ground truth data for the patient user 10 input via one or more I/O units 133 (e.g., feedback from the patient user 10, such as responses to a survey inquiring about experience of the patient user 10 following a dosing session), etc.

In one embodiment, for each of the one or more patient users 10 (e.g., user A, user B, and user C), the training data comprises data tracked/collected by one or more third party user devices 140 associated with a third party user 20 assigned to the patient user 10 such as, but not limited to, ground truth data for the patient user 10 input via one or more I/O units 143 (e.g., observations from the third party user 20, such as observations during patient intake of the patient user 10, observations during a high dose session involving the patient user 10), etc.

In one embodiment, the training data collector 420 maintains, on at least one storage device 152 of the remote computing environment 150, one or more databases comprising the training data.

In one embodiment, the training data collector 420 provides an application programming interface (API) that facilitates collection of the training data from different devices and data sources, including patient user devices 130 and third party user devices 140.

In one embodiment, the AI and machine learning system 410 comprises a feature extractor 430 configured to: (1) receive a portion of the training data (e.g., the sensor data included in the training data) from the training data collector 420, and (2) extract, from the portion of the training data, features for each of the one or more patient users 10 (e.g., user A, user B, and user C). An extracted feature for a patient user 10 may comprise either data in a raw form (e.g., user A had a peak heart rate of 100 beats per minute after a microdosing session) or a specific range bin in which the patient user 10 may be placed (e.g., a 45-year-old patient user 10 may be placed into a range bin for 40-50 year old patient users). In one embodiment, the feature extractor 430 performs feature selection to select only features that would contribute most to the quality of a resulting machine learning model 450 (i.e., eliminating features that are irrelevant and can decrease the accuracy and quality of the model 450). Feature selection includes data correlation.

In one embodiment, the AI and machine learning system 410 comprises a training engine 440 configured to: (1) receive a portion of the training data (e.g., the ground truth data included in the training data) from the training data collector 420, (2) the extracted features from the feature extractor 430, and (3) train, based on the portion of the training data and the extracted features, one or more machine learning models 450 using one or more supervised machine learning techniques.

In one embodiment, a machine learning model 450 maps an extracted feature for a patient user 10 to ground truth data for the patient user 10.

In one embodiment, the training engine 440 is configured to train a machine learning model 450 on a server device 151 of the remote computing environment 150.

In one embodiment, the one or more supervised machine learning techniques include, but are not limited to, Logistic Regression, Decision Tree, Naive Bayes, Support Vector Machine, or Artificial Neural Network.

In one embodiment, a machine learning model 450 may be re-built/updated on an as-needed basis. In one embodiment, a machine learning model 450 may be updated online after new training data is collected by the training data collector 420.

In one embodiment, extracted features for a patient user 10 may be weighted differently in order to emphasize some extracted features more than other extracted features. The weighting of each extracted feature may be based on context and one or more characteristics/requirements of a prescribed therapeutic treatment plan (e.g., desired mental health goal). For example, extracted features relating to mental health of a patient user 10 may be emphasized with a higher weight. The weighting of each extracted feature may additionally be based on impact of the extracted feature in determining experience/satisfaction of a patient user 10 with a prescribed therapeutic treatment plan. For example, if an extracted feature has little to no impact in determining experience/satisfaction of a patient user 10 with a prescribed therapeutic treatment plan, the extracted feature may be de-emphasized with a lower weight.

Each machine learning model 450 resulting from the training phase is used in the deployment phase. For example, in one embodiment, a machine learning model 450 is used in the deployment phase to determine experience/satisfaction of a patient user 10 with a prescribed therapeutic treatment plan. As another example, in one embodiment, a machine learning model 450 is used in the deployment phase to determine a recommendation. In one embodiment, a recommendation comprises a recommended/suggested therapeutic treatment plan prescribed to a patient user 10. A third party user 20 can prescribe a new therapeutic treatment plan to a new patient user 10 based in part on a recommended/suggested therapeutic treatment plan from a machine learning model 450. In one embodiment, a recommendation comprises a recommended/suggested adjustment to an existing therapeutic treatment plan prescribed to an existing patient user 10 (e.g., change the existing therapeutic treatment plan, such as adding a high dose session, increasing/decreasing amount of psilocybin to take/ingest during a microdosing session, etc.).

In one embodiment, a machine learning model 450 resulting from the training phase is specific to a particular demographic/population of patient users 10. For example, a plurality of patient users 10 from which training data will be collected during the training phase are clustered into one or more groups (e.g., groups of similar ages, similar occupations, similar geographical locations, etc.), and at least one machine learning model 450 specific to a group is trained based on training data collected from the group. Each group represents a pool of peers. Feedback from a particular group following dosing sessions is used to finetune/update a machine learning model 450 specific to the group. In one embodiment, each group is assigned a corresponding type of patient user profile.

For example, in one embodiment, a machine learning model 450 may be trained based on training data collected from a set of patient users 10 exhibiting the following characteristics: (i) male, (ii) 30-35 years old, (iii) veteran, (iv) weighs 180-200 lbs, and (iv) exhibits PTSD.

As another example, in one embodiment, a machine learning model 450 may be trained based on training data collected from a set of patient users 10 enrolled in a particular program. The resulting machine learning model 450 may be automatically applied, in the deployment phase, for many patient users 10 enrolled in the same or similar program in a scalable manner.

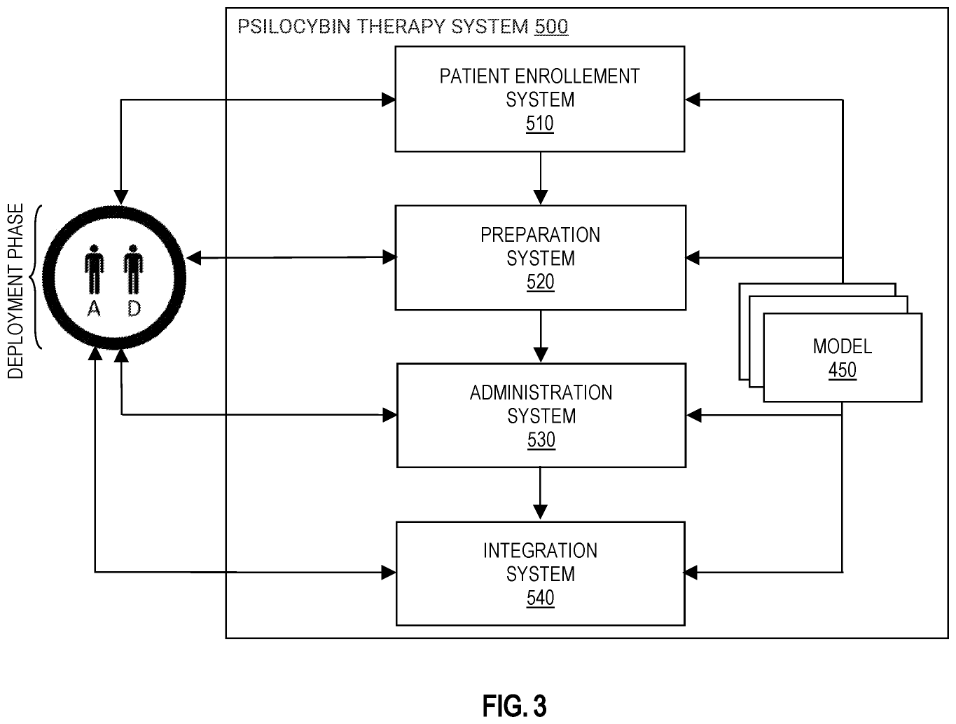

FIG. 3 illustrates one or more example components of the telehealth system 400 utilized in the deployment phase, in one or more embodiments. In one embodiment, the telehealth system 400 comprises a psilocybin therapy system 500 utilized in the deployment phase. In one embodiment, the psilocybin therapy system 500 comprises at least the following: a patient enrollment system 510, a preparation system 520, an administration system 530, and an integration system 540.

As described in detail later herein, the patient enrollment system 510 is configured to perform a patient enrollment session involving enrolling a patient user 10 (e.g., user A) in therapeutic treatment involving psilocybin therapy. The preparation system 520 is configured to perform a preparation session involving a video teleconferencing session between the patient user 10 and a third party user 20 (e.g., user D) to assess a mental health of the patient user 10, diagnose to patient user 10, and prescribe a therapeutic treatment plan to the patient user 10. The administration system 530 is configured to perform an administration session involving at least one third party user 20 (e.g., user D or a different third party user 20) guiding, supervising, and observing the patient user 10 during a high dose session conducted either in-person or virtually. The integration system 540 is configured to perform an integration session involving the patient user 10 providing feedback indicative of their experience and/or effects after each dosing session.

In one embodiment, each of the systems 510, 520, 530, and 540 utilizes at least one machine learning model 450 resulting from the training phase.

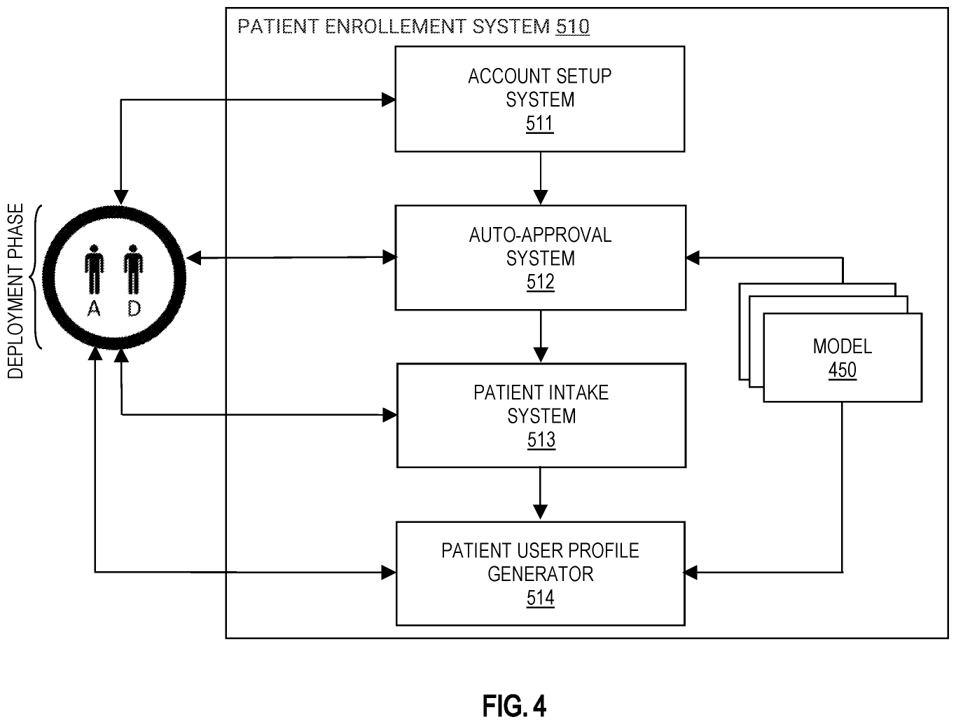

FIG. 4 illustrates one or more example components of the patient enrollment system 510 utilized in the deployment phase, in one or more embodiments. The patient enrollment session is initiated when a patient user 10 (e.g., user A) opens a telehealth app 200 downloaded to a patient user device 130 associated with the patient user 10, and interacts with the telehealth app 200 via one or more I/O units 133 to submit a request to enroll in therapeutic treatment involving psilocybin therapy. For example, in one embodiment, the patient user 10 selects a GUI component in the telehealth app 200 to indicate a desired interest to enroll in therapeutic treatment involving psilocybin therapy, prompting the telehealth app 200 to display one or more GUIs that include a privacy policy and that require the patient user 10 to confirm they are old enough for psilocybin therapy (e.g., at least 21 years of age) and they have read and understood the privacy policy.

In one embodiment, the patient enrollment system 510 comprises an account setup system 511 configured to receive the request from the telehealth app 200 and initiate an account creation process. The account creation process involves the account setup system 511 triggering the telehealth app 200 to display one or more GUIs that prompt the patient user 10 to create an account by providing account information via one or more I/O units 133. In one embodiment, the account creation process optionally involves the patient user 10 making a payment, via the telehealth app 200, for an initial consultation with a third party user 20. In one embodiment, the account creation process involves the patient user 10 selecting a GUI component in the telehealth app 200 to identify whether they are a self-referral or a referral from a third party user 20 (e.g., a healthcare provider).

In one embodiment, a third party user 20 can open a telehealth app 300 downloaded to a third party user device 140 associated with the third party user 20, and interact with the telehealth app 300 via one or more I/O units 143 to create an account and identify as a third party user 20 interested in referring one or more clients for therapeutic treatment involving psilocybin therapy.

In one embodiment, the patient enrollment system 510 comprises an auto-approval system 512 configured to receive confirmation the patient user 10 has created an account from the account setup system 511, and initiate an automatic approval process. The automatic approval process involves the auto-approval system 512 providing the telehealth app 200 a set of auto-approval questions for display on the patient user device 130. In one embodiment, the set of auto-approval questions comprises one or more questions for use in determining urgency of therapeutic treatment for the patient user 10. The automatic approval process involves the patient user 10 interacting with the telehealth app 200 via the one or more I/O units 133 to submit responses to the set of auto-approval questions.

Table 1 below provides an example set of auto-approval questions presented to a patient user 10, via a telehealth app 200.

TABLE 1

ARE YOU A FIRST-RESPONDER (FIREFIGHTER, EMT,
POLICE, ETC )? IF YES, CONTINUE.
ARE YOU A VETERAN WHO HAS EXPERIENCED TRAUMA
OR COMBAT? IF YES, CONTINUE.
HAVE YOU FOUND MEDICATIONS AND PSYCHOTHERAPY
TO BE INEFFECTIVE IN HELPING YOU WITH YOUR MENTAL
HEALTH ISSUES? IF YES, CONTINUE.
HAVE YOU BEEN TREATED FOR MENTAL HEALTH ISSUES?
IF YES, CONTINUE.
DO YOU OFTEN EXPERIENCE STRESS, DEPRESSION OR
ANXIETY? IF YES, CONTINUE.
ARE YOU DEPRESSED OR HAVE A DIAGNOSIS OF
DEPRESSION? IF YES, CONTINUE.
DO YOU HAVE FREQUENT ANXIETY OR HAVE A DIAGNOSIS
OF ANXIETY DISORDER? IF YES, CONTINUE.
DO YOU HAVE PTSD OR HAVE A DIAGNOSIS OF PTSD? IF
YES, CONTINUE.
DO YOU HAVE EATING DISORDERS OR HAVE A DIAGNOSIS
OF EATING DISORDERS? IF YES, CONTINUE.
ARE YOU OBSESSIVE COMPULSIVE OR HAVE A DIAGNOSIS
OF OC DISORDER? IF YES, CONTINUE.
DO YOU HAVE ADDICTIONS
(DRUGS/ALCOHOL/GAMBLING/PORNOGRAPHY) OR HAVE
AN ADDICTION DIAGNOSIS? IF YES, CONTINUE.
ARE YOU ON THE AUTISTIC SPECTRUM OR HAVE A
DIAGNOSIS OF AUTISTIC SPECTRUM DISORDER? IF YES,
CONTINUE.
DO YOU HAVE HIGH BLOOD PRESSURE OR RECEIVE
MEDICATION FOR HIGH BLOOD PRESSURE? IF YES,
CONTINUE.
DO YOU HAVE A DIAGNOSIS OF CARDIOVASCULAR
DISEASE? IF YES, CONTINUE.
DO YOU HAVE BIPOLAR DISORDER OR A DIAGNOSIS OF
BIPOLAR DISORDER? IF YES, CONTINUE.
DO YOU HAVE SCHIZOPHRENIA OR A DIAGNOSIS OF
SCHIZOPHRENIA? IF YES, CONTINUE.
DO YOU HAVE COGNITIVE DECLINE/DEMENTIA OR A
DIAGNOSIS OF COGNITIVE DECLINE/DEMENTIA? IF YES,
CONTINUE.
DO YOU HAVE A TERMINAL ILLNESS OR HAVE A
DIAGNOSIS OF A TERMINAL ILLNESS? IF YES, CONTINUE.

The automatic approval process includes the auto-approval system 512 receiving the responses to the set of auto-approval questions from the telehealth app 200, and automatically determining, utilizing at least one machine learning model 450, whether the patient user 10 is approved for fast tracking status based on the responses. Fast tracking status indicates that the patient user 10 is in urgent need of therapeutic treatment. If the patient user 10 is approved for fast tracking status, an initial consultation with a third party user 20 is expedited (e.g., immediately follows the patient enrollment session). In one embodiment, a machine learning model 450 utilized by the auto-approval system 512 is trained (during the training phase) to determine whether a patient user 10 is eligible/qualifies for fast tracking status based on responses from the patient user 10 to a set of auto-approval questions.

In one embodiment, that automatic approval process is designed to automatically filter patient users 10 seeking mental health treatment from others only interested in psychedelics for recreational purposes.

In one embodiment, the patient enrollment system 510 comprises patient intake system 513 configured to receive information indicative of whether the patient user 10 is approved for fast tracking status from the auto-approval system 512, and initiate a patient intake process online/virtually. The patient intake process involves the patient intake system 513 providing the telehealth app 200 a questionnaire for display on the patient user device 130. In one embodiment, the questionnaire comprises a set of questions relating to well-being of the patient user 10. In one embodiment, the questionnaire is adapted based on at least one of the following: whether the patient user 10 is a self-referral or a referral from a third party user 20, whether the patient user 10 is approved for fast tracking status. The patient intake process includes the patient user 10 interacting with the telehealth app 200 via the one or more I/O units 133 to submit responses to the questionnaire. The patient intake process includes the patient intake system 513 receiving the responses to the questionnaire from the telehealth app 200.

In one embodiment, the same or a similar questionnaire is presented to the patient user 10 at other times during a duration of their therapeutic treatment, such as at one week and four weeks following the patient user 10 completing a dosing session included in a prescribed therapeutic treatment plan.

Table 2 below provides an example questionnaire, presented to a patient user 10 via a telehealth app 200.

TABLE 2
WELL-BEING QUESTIONNAIRE:

```
PLEASE CLICK THE BUTTON WHICH BEST REFLECTS
YOUR CURRENT SITUATION/FREQUENCY
DURING THE LAST 4 WEEKS, HOW OFTEN HAVE YOU
BEEN BOTHERED BY ANY OF THE FOLLOWING? FOR EACH,
CLICK ONE OF THE FOLLOWING BUTTONS: “NOT AT ALL”,
“SOME OF THE TIME”, “A LOT OF THE TIME”, OR “MOST OR
ALL OF THE TIME”
  1. AVOIDING TALKING, PEOPLE, PLACES AND ACTIVITIES
ASSOCIATED WITH TRAUMA
  2. DIFFICULTY CONCENTRATING
  3. FEELING HOPELESS
  4. FEELING SHAME, GUILT
  5. FEELING WORTHLESS
  6. FEEL DETACHED FROM LOVED ONES, FRIENDS,
FAMILY
  7. FREQUENTLY DISLIKE YOURSELF
  8. MEMORY PROBLEMS ASSOCIATED WITH TRAUMATIC
EVENT
  9. UNWANTED, RECURRING & DISTRESSING MEMORIES
10. NIGHTMARES ABOUT TRAUMA
11. DIFFICULTY SLEEPING
12. EASILY STARTLED/FRIGHTENED
13. ALWAYS ″ON GUARD″
14. OUTBURSTS OF ANGER
15. PHYSICAL DISCOMFORTS SUCH AS PAIN, SWEATING,
TREMBLING, TWITCHING, UPSET STOMACH, DIARRHEA,
RAPID BREATHING
16. VIVID FLASHBACKS (FEELING LIKE THE TRAUMA IS
HAPPENING RIGHT NOW)
17. HABIT OF SKIPPING MEALS
18. EATING VERY LITTLE FOOD
19. BINGE EATING
20. EATING IN SECRET
21. FEAR OF GAINING WEIGHT
22. LEAVING THE TABLE TO USE BATHROOM & VOMIT
FOOD
23. USE LAXATIVES, ETC. TO REMOVE FOOD
24. EXCESSIVE EXERCISE
25. OBSESS OVER WEIGHT/SHAPE/SIZE
26. AVOID SOCIALIZING WITH OTHERS
27. RESTLESSNESS, AGITATION OR TENSION
28. NERVOUSNESS, IRRITABILITY OR DREAD
29. FEELING PANICKED OR DANGER
30. DECREASED OR INCREASED APPETITE
31. SLEEPING TOO MUCH OR TOO LITTLE
32. LACK OF PLEASURE IN ACTIVITIES I PREVIOUSLY
ENJOYED
33. FEELING OF SADNESS
34. THOUGHTS OF SUICIDE OR ACTIVE PLAN TO END
ONE'S LIFE
35. LACK OF ENERGY, FATIGUE, TIREDNESS
36. FORGETTING APPOINTMENTS AND DATES
37. FORGETTING RECENT CONVERSATIONS AND
EVENTS
38. FEELING INCREASINGLY OVERWHELMED BY
MAKING DECISIONS AND PLANS
39. HAVING A HARD TIME UNDERSTANDING DIRECTIONS
OR INSTRUCTIONS
40. LOSING YOUR SENSE OF DIRECTION
41. LOSING THE ABILITY TO ORGANIZE TASKS
42. BECOMING MORE IMPULSIVE
43. ANXIETY
44. DEPRESSION
45. NEEDING THINGS ORDERLY AND SYMMETRICAL
46. DOUBTING AND HAVING DIFFICULTY TOLERATING
UNCERTAINTY
47. WASHING AND CLEANING
48. CHECKING
49. UNWANTED THOUGHTS
50. FOLLOWING A STRICT ROUTINE
```

In one embodiment, the questionnaire presented during the patient intake process includes one or more questions inquiring about the experience of the patient user 10 experimenting with psychedelics such as, but not limited to, “Have you experimented with psychedelics and how often?”, “Have you used Psilocybin?”, “Have you used MDMA (Ecstasy/Molly)?”, “Have you used LSD?”, “Have you used Ayahuasca?”, “Have you used Peyote (Mescaline)?”, “Have you used DMT(Fantasia)?”, “Have you used Other Drugs?”, “Have you used Heroin?”, “Have you used Cocaine?”, “Have you used Methamphetamine?”, etc.

If the patient user 10 is a referral from a third party user 20, the patient intake process includes the patient intake system 513 prompting the third party user 20 to export an electronic medical record of the patient user 10 to the patient intake system 513.

In one embodiment, the patient enrollment system 510 maintains, on at least one storage device 152 of the remote computing environment 150, one or more databases comprising Health Insurance Portability and Accountability Act (HIPAA) compliant personal data for a patient user 10, such as account information for an account created for the patient user 10, responses from the patient user 10 to a set of auto-approval questions, responses from the patient user 10 to a questionnaire, an electronic medical record of the patient user 10 imported from a third party user 20, etc.

In one embodiment, the patient enrollment system 510 comprises a patient profile generator 514 configured to receive the responses to the questionnaire from the patient intake system 513, and initiate a patient user profile generation process. The patient user profile generation process includes determining, utilizing at least one machine learning model 450, a type of patient user profile suitable for the patient user 10 based on the responses. In one embodiment, a machine learning model 450 utilized by the patient user profile generator 514 is trained (during the training phase) to determine a type of patient user profile that a patient user 10 fits/classifies into. Examples of different types of patient user profiles include, but are not limited to, a first type for first responders, a second type for veterans, etc. The patient user profile generation process includes the patient intake system 513 generating a patient user profile for the patient user 10 in accordance with the type of patient user profile determined. In one embodiment, the patient user profile is adapted based on at least one of the following: whether the patient user 10 is a self-referral or a referral from a third party user 20, whether the patient user 10 is approved for fast tracking status.

In one embodiment, the patient user profile comprises an electronic medical record populated based on the responses and the type of patient user profile determined, and a set of diagnostic questions to ask and observations to make based on the responses and the type of patient user profile determined. As described in detail later herein, the patient user profile is provided to a third party user 20 (e.g., user D) to prepare for an initial consultation with the patient user 10. The patient user provides a snapshot of the patient user 10, thereby aiding the third party user 20 to more accurately and quickly assess a mental health of the patient user 10 and diagnose the patient user 10. Specifically, the set of diagnostic questions to ask and observations to make allow the third party user 20 to accelerate mental health assessment and mental health diagnosis of the patient user 10. In one embodiment, the set of diagnostic questions to ask and observations to make are customized utilizing AI. For example, in one embodiment, the patient profile generator 514 utilizes AI to select one or more diagnostic questions to ask from a complete list of available diagnostic questions, thereby narrowing down diagnostic questions to ask only to those suitable for/relevant to the patient user 10.

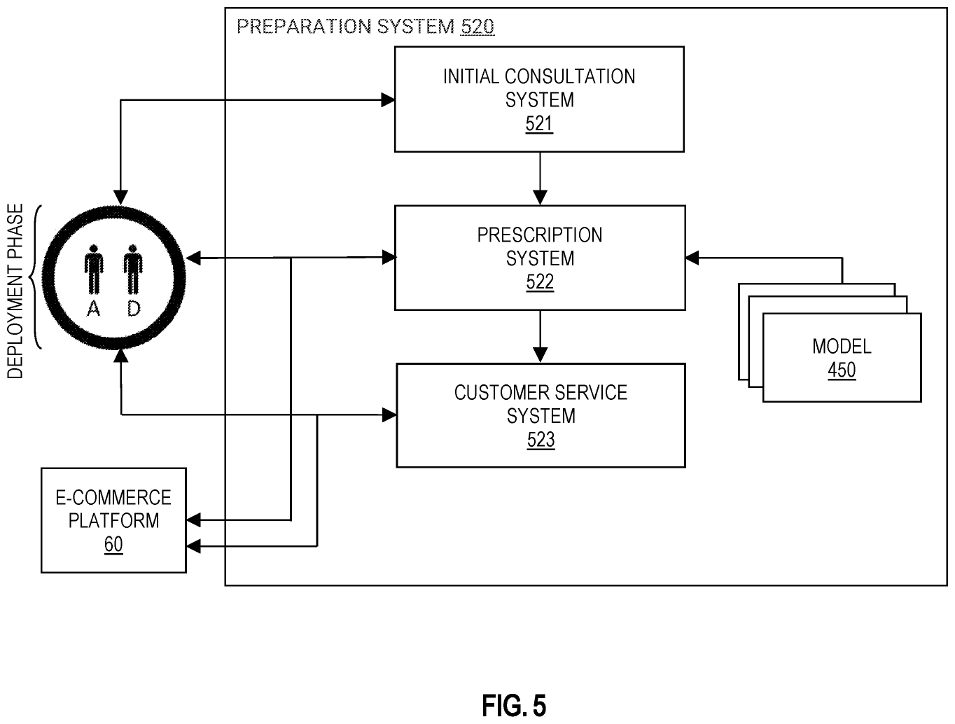

FIG. 5 illustrates one or more example components of the preparation system 520 utilized in the deployment phase, in one or more embodiments. In one embodiment, the preparation system 520 comprises an initial consultation system 521 configured to receive a patient user profile for a patient user 10 from the patient enrollment system 510, and initiate a preparation session. The preparation session involves the initial consultation system 521 scheduling the patient user 10 for an initial consultation with a third party user 20 (e.g., user D) that will take place online/virtually. If the patient user 10 is approved for fast tracking status, the initial consultation system 521 schedules the initial consultation as soon as possible (e.g., on the same day immediately following the patient enrollment session); otherwise, the initial consultation system 521 schedules the initial consultation at a later time/day. The initial consultation system 521 provides an invite to join a video teleconferencing session at the scheduled time for the initial consultation to both a telehealth app 200 on a patient user device 130 associated with the patient user 10 and a telehealth app 300 on a third party user device 140 associated with the third party user 20.

The preparation session involves the initial consultation system 521 providing, to the telehealth app 300, the patient user profile for the patient user 10. The third party user 20 can interact with the telehealth app 300 via one or more I/O units 143 to review the patient user profile.

The preparation session involves the video teleconferencing session at the scheduled time for the initial consultation (e.g., immediately after the patient enrollment session or at a later time/day). The initial consultation starts when both the patient user 10 and the third party user 20 join the video teleconferencing session via the telehealth app 200 and the telehealth app 300, respectively. In one embodiment, the initial consultation system 521 provides, to the telehealth app 200/300, an alert/notification reminding the user 10/20 of the scheduled time for the initial consultation.

During the initial consultation, the initial consultation system 521 is configured to: (1) provide, to the telehealth app 200, real-time video of the third party user 20 for display on the patient user device 130, and (2) provide, to the telehealth app 300, real-time video of the patient user 10 and the patient user profile for simultaneous display on the third party user device 140. For example, in one embodiment, the third party user 20 views a split screen of the patient user profile and the real-time video of the patient user 10 on the third party user device 140, while the patient user 10 views the real-time video of the third party user 20.

During the initial consultation, the third party user 20 asks the patient user 10 one or more diagnostic questions and/or makes one or more observations in accordance with the patient user profile (displayed, via the telehealth app 300, on the third party user device 140). The patient user profile enables the third party user 20 to more accurately and quickly assess the mental health of the patient user 10 and diagnose the patient user 10. During the initial consultation, the third party user 20 interacts with the telehealth app 300 via the one or more I/O units 143 to submit a mental health assessment and a mental health diagnosis of the patient user 10. The assessment includes each response from the patient user 10 to each diagnostic question asked and each observation made by the third party user 20 during the initial consultation. In one embodiment, an electronic medical record for the patient user 10 is updated/annotated to include the mental health assessment and the mental health diagnosis.

Table 3 below provides an example set of diagnostic questions to ask and observations to make during patient intake, presented to a third party user 20 via a telehealth app 300.

TABLE 3

```
I. Appearance (Observed) - Possible descriptors:
  Gait, posture, clothes, grooming.
II. Behavior (Observed) - Possible descriptors:
  Mannerisms, gestures, psychomotor activity, expression, eye
contact, ability to follow commands/requests, compulsions.
III. Attitude (Observed) - Possible descriptors:
  Cooperative, hostile, open, secretive, evasive, suspicious,
apathetic, easily distracted, focused, defensive.
IV. Level of Consciousness (Observed) - Possible descriptors:
  Vigilant, alert, drowsy, lethargic, stuporous, asleep, comatose,
confused, fluctuating.
V. Orientation (Inquired) - Possible questions for patient:
  “What is your full name?”
  “Where are we at (floor, building, city, county, and state)?”
  “What is the full date today (date, month, year, day of the week,
and season of the year)?”
  “How would you describe the situation we are in?”
VI. Speech and Language (Observed)
A. Quantity - Possible descriptors:
  Talkative, spontaneous, expansive, paucity, poverty.
B. Rate - Possible descriptors:
  Fast, slow, normal, pressured.
C. Volume (Tone) - Possible descriptors:
  Loud, soft, monotone, weak, strong.
D. Fluency and Rhythm - Possible descriptors:
  Slurred, clear, with appropriately placed inflections, hesitant,
with good articulation, aphasic.
VII. Mood (Inquired): A sustained state of inner feeling - Possible
questions for patient:
  “How are your spirits?”
  “How are you feeling?”
  “Have you been discouraged/depressed/low/blue lately?”
  “Have you been energized/elated/high/out of control lately?”
  “Have you been angry/irritable/edgy lately?”
VIII. Affect (Observed): An observed expression of inner feeling. -
Possible descriptors:
  Appropriateness to situation, consistency with mood, congruency
with thought content.
  Fluctuations: Labile, even.
  Range: Broad, restricted.
  Intensity: Blunted, flat, normal intensity.
  Quality: Sad, angry, hostile, indifferent, euthymic, dysphoric,
detached, elated, euphoric, anxious, animated, irritable.
IX. Thought Processes or Thought Form (Inquired/Ob served):
logic, relevance, organization, flow and coherence of
thought in response to general questioning during the interview. -
Possible descriptors:
  Linear, goal-directed, circumstantial, tangential, loose
associations, incoherent, evasive, racing, blocking, perseveration,
neologisms.
X. Thought Content (Inquired/Observed) - Possible questions for
patient:
  “What do you think about when you are sad/angry?”
  “What’s been on your mind lately?”
  “Do you find yourself ruminating about things?”
  “Are there thoughts or images that you have a really difficult
time getting out of your head?”
  “Are you worried/scared/frightened about something or other?”
  “Do you have personal beliefs that are not shared by others?”
(Delusions are fixed, false, unshared beliefs.)
  “Do you ever feel detached/removed/changed/different from
others around you?”
  “Do things seem unnatural/unreal to you?”
  “What do you think about the reports in papers such as The
National Enquirer?”
  “Do you think someone, or some group intend to harm you in
some way?”
  [In response to something the patient says] “What do you think
they meant by that?”
  “Does it ever seem like people are stealing your thoughts, or
perhaps inserting thoughts into your head? Does it ever seem like
your own thoughts are broadcast out loud?”
  “Do you ever see (visual), hear (auditory), smell (olfactory),
taste (gustatory), and feel (tactile) things that are not really there,
such as voices or visions?” (Hallucinations are false perceptions)
  “Do you sometimes misinterpret real things that are around you,
such as muffled noises or shadows?”
(Illusions are misinterpreted perceptions)
XI. Suicidality and Homicidality
A. Suicidality - Possible questions for patient:
  “Do you ever feel that life isn’t worth living? Or that you would
just as soon be dead?”
  “Have you ever thought of doing away with yourself? If so,
how?”
  “What would happen after you were dead?”
B. Homicidality - Possible questions for patient:
  “Do you think about hurting others or getting even with people
who have wronged you?”
  “Have you had desires to hurt others? If so, how?”
XII. Insight and Judgment (Inquired/Ob served) - Possible questions
for patient:
  “What brings you here today?”
  “What seems to be the problem?”
  “What do you think is causing your problems?”
  “How do you understand your problems?”
  “How would you describe your role in this situation?”
  “Do you think that these thoughts, moods, perceptions, are
abnormal?”
  “How do you plan to get help for this problem?”
  “What will you do whenoccurs?”
  “How will you manage ifhappens?”
  “If you found a stamped, addressed envelope on the street, what
would you do with it?”
  “If you were in a movie theater and smelled smoke, what would
you do?”
XIII. Attention (Inquired/Ob served) - Possible descriptors:
  Attend, concentration, distractibility.
A. Digit Span (forward and reverse) - Suggested patient instructions:
  “I will recite a series of numbers to you, and then I will ask you to
repeat them to me, first forwards and then backwards.”
[Begin with 3 numbers - not consecutive numbers, and advance to
7-8 numbered sequence.]
B. Spelling Backwards - Suggested patient instructions:
  “Spell the word ‘world.’ Now spell the word ‘world’ backwards.”
C. Calculations - Suggested patient instructions:
  (Serial 7’s) “Starting with 100, subtract 7 from 100, and then
keep subtracting 7 from that number as far as you can go.”
  (Serial 3’s) “Starting with 20, subtract 3 from 20, and then keep
subtracting 3 from that number as far as you can go.” [Monitor for
speed, accuracy, effort required, and monitor patient reactions to
the request]
  “Add these numbers: (15 + 12 + 7)”
  “Multiply these numbers: (25 × 6)”
  “If something costs 78 cents and you give the cashier one dollar,
how much change should you get back?”
XIV. Memory (Inquired)
A. Recent Memory - Possible questions for patient:
  “What is my name?”
  “What medications did you take today?”
  “What time was your appointment with me for today?”
B. Remote Memory - Possible questions for patient:
  “Where were you when President Kennedy was shot?” (For
patients over 40)
  “What is your Social Security number?”
  “What were the dates of your graduation from high school,
college, graduate school?”
  “When and where did you get married?”
C. Immediate Memory (also see XIII.-A. above) and New
Learning - Suggested patient instructions:
  “I am going to ask you to remember three words (color,
object, animal - e.g., blue, table, and horse)
and I will ask you to repeat them to me in 5 minutes. Please
repeat them now after me: blue, table, and horse.”
- 5 minutes elapse - “What were those three words I asked you
to remember?” [Monitor accuracy of response, awareness of
whether responses are correct, tendency to confabulate or
substitute other words, ability to correct themselves with
category clue and multiple choice].
XV. Intellectual (Inquired/Observed)
A. Information and Vocabulary - Suggested patient instructions:
  “Name the last 5 presidents.” (Clinton, Bush, Reagan, Carter,
Ford, Nixon, ...)
  “Name 5 of the largest cities in the country.” (New York City,
Los Angeles, Chicago, Houston, Philadelphia)
  “Name the current president, vice president, governor, and
mayor.” (Bill Clinton, Al Gore, George Ryan, Richard M. Daley)
B. Vocabulary - Possible descriptors:
  Grade school level, high school level, fluent, consistent with
education.
C. Abstraction - Possible questions for patient:
1. Similarities - “How are the following items similar?”
  “an apple and an orange” (round ~concrete, fruit ~abstract)
  “a chair and a table” (made of wood ~concrete, furniture
~abstract)
  “a watch and a ruler” (measurement instruments ~abstract)
2. Proverbs - “How would you describe the meaning of the
following sayings?”
  “People living in glass houses should not throw stones.”
  “A bird in the hand is worth two in the bush.”
  “You shouldn’t cry over spilt milk.”
  “Two heads are better than one.”
```

The initial consultation allows the third party user 20 to get to know the patient user 10 and form a trusting relationship with the patient user 10. This helps the patient user 10 relax and feel assured that there will be professionals advocating for and supporting them during therapeutic treatment. The initial consultation enables the third party user 20 to determine motivation and preparedness of the patient user 10 participate in psilocybin therapy, prior experiences of the patient user 10 with conventional therapies (e.g., medication and/or psychotherapy), suicide risk, and/or a DSM-V (Diagnostic and Statistical Manual of Mental Disorders, Fifth Edition) diagnosis.

During the initial consultation, the third party user 20 educates the patient user 10 on psilocybin, microdosing, psychedelic-assisted therapy, the range of psilocybin experiences and side effects as well as safety considerations. During the initial consultation, the third party user 20 can answer questions the patient user 10 may have.

In one embodiment, as part of educating the patient user 10 on psychedelic-assisted therapy including psilocybin therapy, the initial consultation system 521 provides, to the telehealth app 200, one or more educational videos for presentation on the patient user device 130 (e.g., educational videos on microdosing and/or psychedelic-assisted therapy). The patient user 10 is required to view each educational video provided and complete knowledge and comprehension checks within each component of the educational video prior to advancing forward to viewing additional information and materials (e.g., another educational video). In one embodiment, an electronic medical record for the patient user 10 is updated/annotated to identify which educational videos the patient user 10 has viewed and completed knowledge and comprehension checks for.

In one embodiment, the initial consultation system 521 is configured to record audio and/or video of the initial consultation, resulting in an audio and/or video recording of the initial consultation. In one embodiment, an electronic medical record for the patient user 10 is updated/annotated to include an audio and/or video recording of an initial consultation the patient user 10 joined.

In one embodiment, the preparation system 520 includes a prescription system 522 configured to receive the mental health assessment and the mental health diagnosis of the patient user 10 from the initial consultation system 521, and initiate a prescription process. The prescription process involves the third party user 20 prescribing a therapeutic treatment plan to the patient user 10 based on the mental health assessment and the mental health diagnosis. In one embodiment, the therapeutic treatment plan is prescribed during the initial consultation. In another embodiment, the therapeutic treatment plan is prescribed after the initial consultation. The prescription process involves the prescription system 522 providing, to the telehealth app 200, an explanation of the therapeutic treatment plan for display on the patient user device 130. The prescription process involves the patient user 10 interacting with the telehealth app 200 via the one or more I/O units 133 to review the therapeutic treatment plan and confirm their understanding of, commitment to participate in, and approval of therapeutic treatment plan.

In one embodiment, an electronic medical record for the patient user 10 is updated/annotated to include a prescribed therapeutic treatment plan.

In one embodiment, the therapeutic treatment plan identifies one or more types of dosing sessions the patient user 10 is expected to complete, frequency of the dosing sessions, and amount of psilocybin to take/ingest during each dosing session. For example, the therapeutic treatment plan may include only microdosing sessions, only high-dose psychedelic-assisted therapy sessions, or a combination of the two.

In one embodiment, the prescription process involves the prescription system 522 determining, utilizing at least one machine learning model 450, a recommendation based on the mental health assessment and the mental health diagnosis of the patient user 10 and, optionally, the type of patient user profile the patient user 10 fits/classifies into, wherein the recommendation comprises a recommended/suggested therapeutic treatment plan to prescribe to the patient user 10. The prescription process involves the prescription system 522 providing, to the telehealth app 300, the recommendation for display on the third party user device 140. The prescription process involves the third party use 20 interacting with the telehealth app 300 via the one or more I/O units 143 to review the recommendation. In one embodiment, the third party user 20 prescribes the therapeutic treatment plan to the patient user 10 further based on the recommendation. For example, in one embodiment, the therapeutic treatment plan prescribed to the patient user 10 is identical to the recommendation or similar to the recommendation. In one embodiment, a machine learning model 450 utilized by prescription system 522 is trained (during the training phase) to determine a recommended/suggested therapeutic treatment plan to prescribe to the patient user 10 based on the mental health assessment and the mental health diagnosis and, optionally, the type of patient user profile the patient user 10 fits/classifies into.

In one embodiment, the preparation system 520 includes a customer service system 523 configured to receive the therapeutic treatment plan prescribed for the patient user 10 from the prescription system 522, and assign a different third party user 30 to act as a customer service representative for the patient user 10. In one embodiment, the customer service representative will contact the patient user 10 to confirm their understanding of, commitment to participate in, and approval of therapeutic treatment plan. The customer service representative will create a unique patient identification number for the patient user 10, place orders for one or more services (e.g., high dose session) and/or one or more products (e.g., psilocybin) required as part of the therapeutic treatment plan, manage billing for the patient user 10, schedule dosing sessions included in the therapeutic treatment plan, and/or assign at least one other third party user 20 to guide, supervise, and observe the patient user 10 during a high dose session. The customer service system 523 provides, via the telehealth app 200, the unique patient identification number, an identification card including the number for reference by the patient user 10, and/or a summarization of information and materials on microdosing and/or psychedelic-assisted therapy that the patient user 10 was educated about during the initial consultation.

In one embodiment, the customer service system 523 is configured to: (1) provide communications to the patient user 10 such as, but not limited to, emails and/or alerts/notifications of scheduled/upcoming dosing sessions for the patient user 10, requests for the patient user 10 to provide feedback describing their experience/satisfaction with a completed dosing session, etc., and (2) provide communications to a third party user 20 assigned to the patient user 10 such as, but not limited to, emails and/or alerts/notifications of scheduled/upcoming high dose sessions the third party user 20 will guide, supervise, and observe, recommendations to adjust a prescribed therapeutic treatment plan that the third party user 20 is assigned to monitor, etc.

In one embodiment, an electronic medical record for the patient user 10 is updated/annotated to include communications provided to the patient user 10 and, optionally, communications provided to a third party user 20 assigned to the patient user 10.

A microdosing session involves the patient user 10 microdosing in their own environment (e.g., home) without supervision of a third party user 20. During a microdosing session, a patient user 10 will orally take/ingest a prescribed amount of psilocybin. The patient user 10 is recommended to drink a large amount of water after taking/ingesting the psilocybin. In one embodiment, an amount of psilocybin prescribed to the patient user 10 to orally take/ingest during a microdosing session is low dose, such as 0.1 grams, 0.2 grams, 0.3 grams, or 0.4 grams. In one embodiment, microdosing sessions are scheduled to be completed every few days (e.g., every three days).

A high dose session involves the patient user 10 orally taking/ingesting a prescribed amount of psilocybin under the guidance, supervision, and observation of at least one third party user 20 assigned to the patient user 10 (e.g., a therapist and a guide). In one embodiment, a guide is a friend or family member of the patient user 10 who is trained as a guide. A high dose session can take place in-person at a designated healthcare facility or virtually via video teleconferencing. For example, in one embodiment, the at least one third party user 20 (present in person or virtually) instructs the patient user 10 to lie down, and provides guidance and reassurance while the patient user 10 orally takes/ingests the psilocybin. The patient user 10 is recommended to drink a large amount of water after taking/ingesting the psilocybin. As the patient user 10 is having a psychedelic experience, the patient user 10 may listen to music and/or wear an eye mask to help them focus internally. The psychedelic experience may last a few hours (e.g., five to six hours), and the at least one third party user 20 is present throughout to make observations of the patient user 10. In one embodiment, an amount of psilocybin prescribed to the patient user 10 to orally take/ingest during a high dose session is high dose, such as 1.0 mg, 5.0 mg, 10.0 mg and 25.0 mg.

In one embodiment, the prescription system 522 and/or the customer service system 523 is configured to exchange data with an e-commerce platform 60 that provides psilocybin dispensary processing and fulfillment services. In one embodiment, the e-commerce platform 60 is operated by the same entity operating the telehealth system 400. In another embodiment, the e-commerce platform 60 is operated by a third party. In one embodiment, the prescription system 522 and/or the customer service system 523 is configured to generate an electronic psilocybin prescription event corresponding to a dosing session included in a therapeutic treatment plan prescribed to a patient user 10. In one embodiment, if the dosing session is a microdosing session, the electronic psilocybin prescription event is communicated (e.g., via email and/or an alert/notification) to either the patient user 10 or a third party user 20 tasked with monitoring experience/satisfaction of the patient user 10 with the therapeutic treatment plan (e.g., a practitioner); otherwise, if the dosing session is a high dose session, the electronic psilocybin prescription event is communicated (e.g., via email and/or an alert/notification) to a third party user 20 tasked with guiding, supervising, and observing the patient user 10 during the high dose session. When a third party user 20 or the patient user 10 interacts with the electronic psilocybin prescription event (e.g., clicking on a link included in an email), an e-commerce transaction is initiated with the e-commerce platform 60 to process and fulfill a prescribed amount of psilocybin for the dosing session, in accordance with the therapeutic treatment plan. If the dosing session is a microdosing session, the prescribed amount is distributed directly to the patient user 10 to self-administer during the microdosing session; otherwise, if the dosing session is a high dose session, the prescribed amount is distributed to the third party user 20 to administer to the patient user 10 during the high dose session.

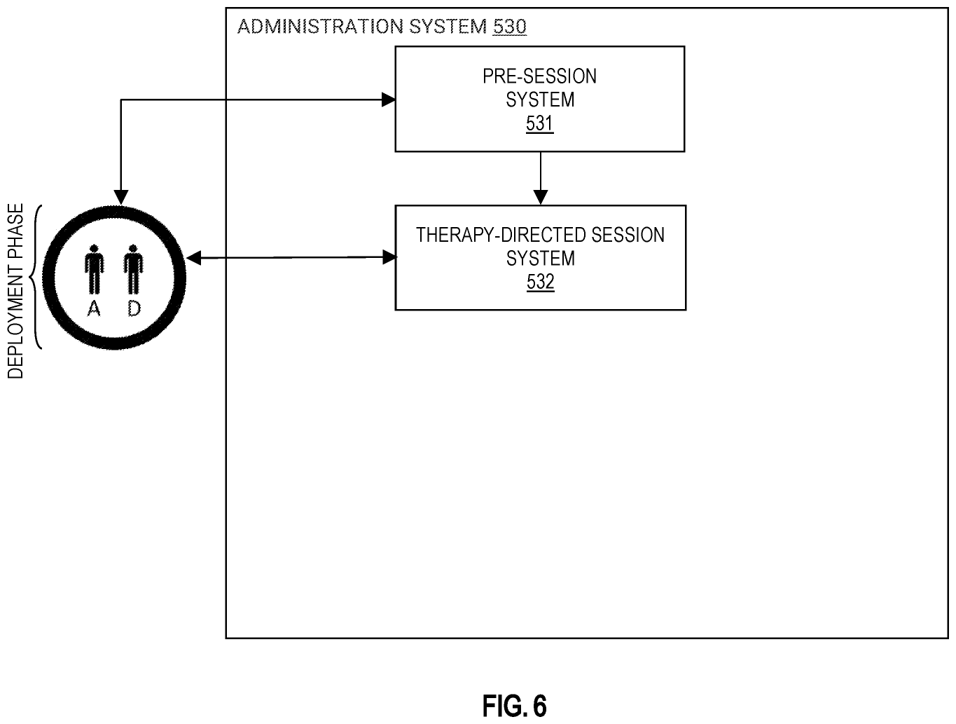

FIG. 6 illustrates one or more example components of the administration system 530 utilized in the deployment phase, in one or more embodiments. In one embodiment, the administration system 530 comprises a pre-session system 531 configured to: (1) receive a therapeutic treatment plan prescribed to a patient user 10 from the preparation system 520, wherein the therapeutic treatment plan includes a high dose session, and (2) in advance of the high dose session, schedule and conduct one or more pre-sessions (e.g., two fifty-minute sessions) to prepare the patient user 10 for the high dose session. Each pre-session is conducted in-person or online/virtually via video teleconferencing. Each pre-session involves the patient user 10 and at least one third party user 20 assigned to guide, supervise, and observe the patient user 10 during the high dose session. The purpose of each pre-session is to develop rapport and trust between the patient user 10 and the at least one third party user 20, thereby helping to reduce reactions of fear or anxiety from the patient user 10 during the high dose session. During each pre-session, the life history and current life of the patient user 10 is reviewed, and intentions and expectations for the high dose session is explored. During each pre-session, the at least one third party user 20 educates the patient user 10 on psilocybin, microdosing, psychedelic-assisted therapy, the range of psilocybin experiences and side effects as well as safety considerations. During each pre-session, the at least one third party user 20 can answer questions the patient user 10 may have.

In one embodiment, an electronic medical record for the patient user 10 is updated/annotated to include records/notes from a third party user 10 during a pre-session with the patient user 10.

In one embodiment, the administration system 530 comprises a therapy-directed session system 532 configured to conduct the high dose session at the scheduled time. The therapy-directed session system 532 is configured to provide, to a telehealth app 300 on a third party user device 140 associated with the at least one third party user 20, a set of observations for the at least one third party user 20 to make during the high dose session. During the high dose session, the at least one third party user 20 interacts with the telehealth app 300 via one or more I/O units 143 to submit each observation made by the at least one third party user 20 in accordance with the set of observations. In one embodiment, an electronic medical record for the patient user 10 is updated/annotated to include observations made by at least one third party user 20 during a high dose session with the patient user 10.

Table 4 below provides an example set of observations to make during a high dose session, presented to a third party user 20 via a telehealth app 300.

TABLE 4

```
HIGH DOSE PSILOCYBIN IN-SESSION OBSERVATION:
THE CLIENT IS:
PLEASE RATE FOR INTENSITY AND FREQUENCY.
ATTITUDE-
  COOPERATIVE:
  HOSTILE:
  PARANOIA/SUSPICIOUS:
  OPEN:
  FOCUSED:
  DEFENSIVE:
  EASILY DISTRACTED:
  SECRETIVE/CLOSED:
  APATHETIC:
  EVASIVE:
BEHAVIOR-
  TALKING:
  YAWNING:
  RESTLESSNESS:
  GESTURES:
  MANNERISMS:
  NAUSEA/VOMITING:
  TEARING/CRYING:
  LAUGHING:
  SMILING:
LEVEL OF CONSCIOUSNESS-
  STIMUL ATED/AROUSED:
  VISUAL CHANGES:
  FEELINGS OF UNREALITY:
  SLEEPY/DROWSY:
  VIGILANT:
  ALERT:
MOOD-
  PEACEFULNESS:
  ANIMATED:
  SADNESS:
  ANXIOUSNESS:
  ANGER:
  ELATED:
  HAPPY:
  EUTHYMIC:
  DYSPHORIC:
  AGITATION/ IRRITABLE
  LABILE EMOTIONS:
  EVEN, LEVEL EMOTIONS:
  EUPHORIA:
THOUGHTS-
  CONFUSION:
  RACING THOUGHTS:
  LOOSE ASSOCIATIONS:
  TANGENTIAL ASSOCIATIONS:
  INCOHERENCE:
  LOGICAL THOUGHTS:
  OBSESSIVE THOUGHTS:
SPEECH & LANGUAGE-
  TALKATIVE:
  SPONTANEOUS:
  POVERTY:
  FAST:
  SLOW:
  NORMAL:
  PRESSURED:
  LOUD:
  SOFT:
  MONOTONE:
  WEAK:
  STRONG:
  SLURRED:
  CLEAR:
  GOOD ARTICULATION:
```

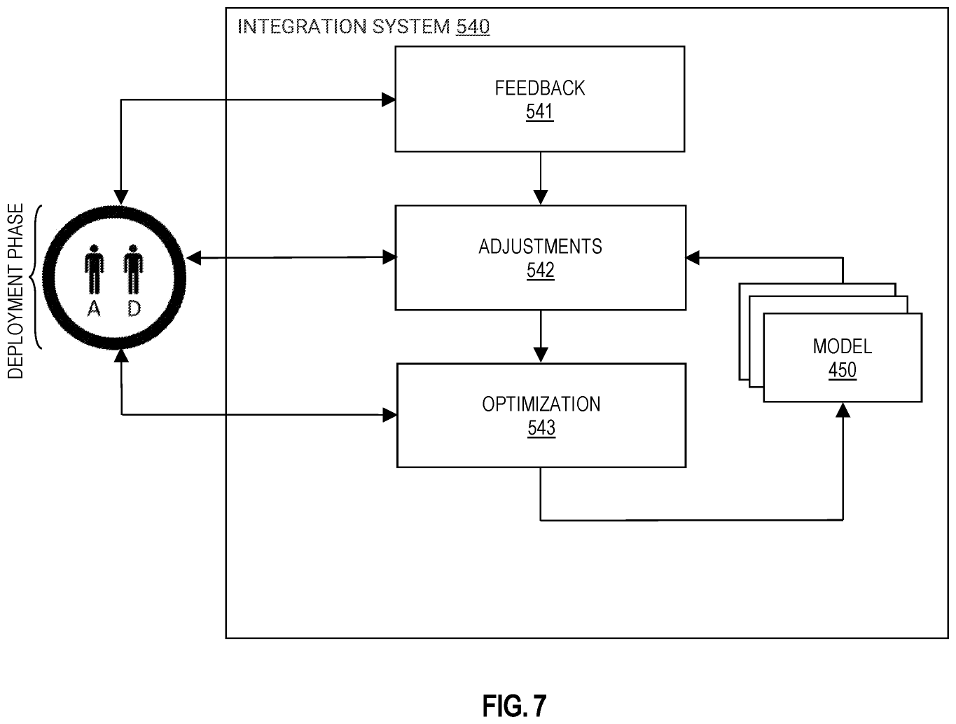

FIG. 7 illustrates one or more example components of the integration system 540 utilized in the deployment phase, in one or more embodiments. In one embodiment, the integration system 540 comprises a feedback system 541 configured to request a patient user 10 to provide feedback indicative of their experience and/or effects after each dosing session.

In one embodiment, the feedback system 541 is configured to request a patient user 10 to provide feedback indicative of their effects after a microdosing session by sending an email and/or alert/notification to the patient user 10 for the patient user 10 to provide a self-report of benefits and challenges they believe they experienced as a result of the microdosing session.

In one embodiment, the feedback system 541 is configured to request a patient user 10 to provide feedback indicative of their experience and/or effects after a high dose session by sending emails and/or alerts/notifications to the patient user 10 for the patient user 10 to: (1) write and submit a short narrative description of their psychedelic experience during the high dose session, wherein the short narrative is based on a set of integration questions presented to the patient user 10 (e.g., via email or the telehealth app 200), (2) provide a self-report of benefits & challenges they believe they experienced as a result of the high dose session, and (3) provide responses to a questionnaire (e.g., see Table 2) comprising a set of questions relating to their well-being. Requiring the patient user 10 to write the short narrative aids the patient user 10 to be more introspective.

For example, in one embodiment, self-reports and/or responses to the questionnaire are requested at the following pre-determined intervals: four weeks, 3 months, 6 months, and 9 months.

Table 5 below provides an example set of integration questions that a patient user 10 utilizes to write a short narrative description of their psychedelic experience during a high dose session.

TABLE 5
INTEGRATION QUESTIONS:

1. Please write a narrative about your psilocybin experience.
It may help you to think of this like telling someone else a
story which communicates your journey through a series
of events. You might think of this like writing a story which
would be made into a movie.
Please include the sequence of events as well as your
thoughts, feelings, what you saw, heard, remembered and
other sensations. This is the, ″What happened″?
2. Please assess your psilocybin story. What meaning does
it have for you? You may want to think of this question as,
″So what″?
3. ″Now what″? What do you think you should do with your
experience? What action should or might you take?

In one embodiment, the integration system 540 is configured to track psilocybin usage of the patient user 10 and compliance of the patient user 10 with a prescribed therapeutic treatment plan to ensure that their therapeutic treatment plan is completed.

In one embodiment, an electronic medical record for the patient user 10 is updated/annotated to include all feedback and responses to the questionnaire received from the patient user 10.

In one embodiment, the integration system 540 comprises an adjustments system 542 is configured to: (1) determine one or more changes in the mental health (e.g., problematic experiences and behavior) of the patient user 10 based on feedback and/or responses to the questionnaire received from the patient user 10, (2) determine a satisfaction level of the patient user 10 with a prescribed therapeutic treatment plan based on the feedback, the responses to the questionnaire, and/or the one or more changes, and (3) request a third party user 10 to make an adjustment to the therapeutic treatment plan in response to determining the satisfaction level is below a pre-determined threshold. A satisfaction level is a metric representing satisfaction of a patient user 10 with a prescribed therapeutic treatment plan. For example, a satisfaction level of a patient user 10 is low if the patient user 10 indicates they are experiencing problematic experiences and behavior.

In one embodiment, the integration session involves the adjustments system 542 determining, utilizing at least one machine learning model 450, a satisfaction level of the patient user 10 based on the feedback, the responses to the questionnaire, and/or the one or more changes. In one embodiment, a machine learning model 450 utilized by the adjustments system 542 is trained (during the training phase) to determine a satisfaction level of the patient user 10 based on the feedback, the responses to the questionnaire, and/or the one or more changes.

In one embodiment, the integration session involves the adjustments system 542 determining, utilizing at least one machine learning model 450, a recommendation based on the feedback, the responses to the questionnaire, the one or more changes, and/or the satisfaction level, wherein the recommendation comprises a recommended/suggested adjustment to the prescribed therapeutic treatment plan. The integration session involves the prescription system 522 providing, to the telehealth app 300, the recommendation for display on the third party user device 140. The integration process involves the third party user 20 interacting with the telehealth app 300 via one or more I/O units 143 to review the recommendation. In one embodiment, the third party user 20 makes an adjustment to the prescribed therapeutic treatment plan based on the recommendation. For example, in one embodiment, the adjustment is identical to the recommendation or similar to the recommendation. In one embodiment, a machine learning model 450 utilized by adjustments system 542 is trained (during the training phase) to determine a recommended/suggested adjustment to the prescribed therapeutic treatment plan based on the feedback, the responses to the questionnaire, the one or more changes, and/or the satisfaction level.

For example, if the patient user 10 indicates they experienced visual effects after a dosing session, the patient user 10 may have taken a too high of a dose, and the third party user 20 adjusts a prescribed therapeutic treatment plan by lowering a prescribed amount of psilocybin that the patient user 10 should take/ingest during a dosing session. Further, orders for psilocybin placed on behalf of the patient user 10 are adjusted accordingly. This process repeats until the patient user 10 indicates they no longer experience visual effects.

In one embodiment, the integration system 540 comprises an optimization system 543 that is configured to perform an optimization process to update/finetune AI and machine learning models 450 utilized by the telehealth system 400 based on all feedback and responses to the questionnaire received from the patient user 10. The optimization system 543 supports a feedback look for the telehealth system 400. For example, if the feedback indicates that conducting a microdosing session one day later than scheduled results in unwanted effects, at least one machine learning model 450 utilized by the telehealth system 400 to determine frequency of microdosing sessions is updated. As another example, if the feedback indicates that a prescribed amount of psilocybin results in unwanted effects, at least one machine learning model 450 utilized by the telehealth system 400 to determine a recommendation for the type of patient user profile the patient user 10 fits/classifies into is updated. Feedback from patient users 10 that fit/classify into a particular type of patient user profile is used to finetune/update a machine learning model 450 specific to the type of patient user profile, thereby improving accuracy and quality of the machine learning model 450.

In one embodiment, the optimization system 543 and/or the AI and machine learning system 410 is utilized to evaluate any correlations between microdosing and overall health of the patient user 10.

In one embodiment, the telehealth system 400 is configured to schedule and conduct one or more follow-up sessions. Each follow-up session is conducted either in-person or online/virtually via video teleconferencing. Each follow-up session involves a patient user 10 and at least one third party user 20 assigned to the patient user 10 to monitor experience/satisfaction of the patient user 10 with a prescribed therapeutic treatment plan. The purpose of each follow-up session is to determine whether the patient user 10 is satisfied with the therapeutic treatment plan and making progress toward a desired mental health goal. During each follow-up session, self-reports from the patient user 10 are reviewed, and the at least one third party user 20 uses integration therapy and cognitive behavioral therapy to help the patient user 10 introspect, process thoughts and feelings, apply and give meaning to their psychedelic experience as well as develop personal purpose and direction to take personal responsibility for making changes in their lives.

For example, in one embodiment, follow-up sessions are conducted at the following pre-determined intervals: one day after a high dose session, four weeks after the high dose session, and every four months.

In one embodiment, an electronic medical record for the patient user 10 is updated/annotated to include records/notes from a third party user 10 during a follow-up session with the patient user 10.

In one embodiment, an electronic medical record for the patient user 10 includes all feedback received from the patient user 10, all responses received from the patient user 10, certifications of all third party users 20 assigned to the patient user 10, and records/notes of all sessions between the patient user 10 and the third party users 20.

In one embodiment, the telehealth system 400 provides healthcare education and training (e.g., certification training programs for third party users 20, educational videos on microdosing and/or psychedelic-assisted therapy, etc.) via video conferencing.

In one embodiment, the telehealth system 400 provides group therapy sessions (e.g., sessions involving patient users 10 classified into the same type of patient user profile, i.e., pool of peers) via video conferencing. Such sessions enable ratings of experiences of patient users 10 in a pool of their peers, thereby facilitating peer-to-peer growth.

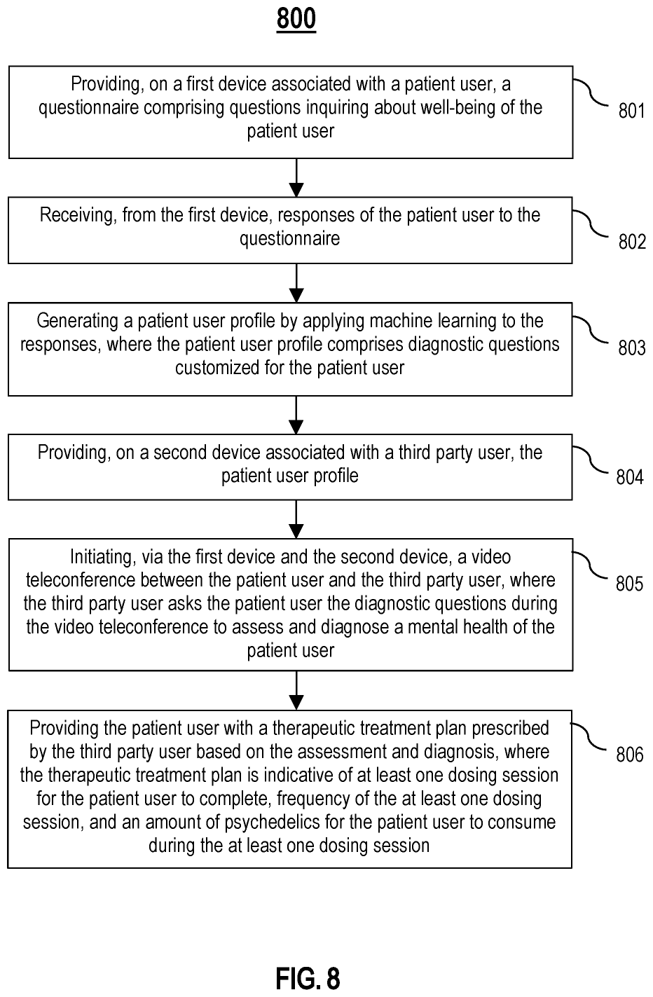

FIG. 8 is a flowchart of an example process 800 for implementing a telehealth platform for psychedelic-assisted therapy, in one or more embodiments. Process block 801 includes providing, on a first device (e.g., a patient user device 130) associated with a patient user (e.g., a patient user 10), a questionnaire comprising questions inquiring about well-being of the patient user. Process block 802 includes receiving, from the first device, responses of the patient user to the questionnaire. Process block 803 includes generating a patient user profile by applying machine learning (e.g., a machine learning model 450) to the responses, where the patient user profile comprises diagnostic questions customized for the patient user.

Process block 804 includes providing, on a second device (e.g., a third party user device 140) associated with a third party user (e.g., a third party user 20), the patient user profile. Process block 805 includes initiating, via the first device and the second device, a video teleconference between the patient user and the third party user, where the third party user asks the patient user the diagnostic questions during the video teleconference to assess and diagnose a mental health of the patient user (i.e., provide tele-mental healthcare). Process block 806 includes providing the patient user with a therapeutic treatment plan prescribed by the third party user based on the assessment and diagnosis, where the therapeutic treatment plan is indicative of at least one dosing session for the patient user to complete, frequency of the at least one dosing session, and an amount of psychedelics for the patient user to consume during the at least one dosing session.

In one embodiment, process blocks 801-806 may be performed by at least one or more of the telehealth system 400, the telehealth app 200, and/or the telehealth app 300.

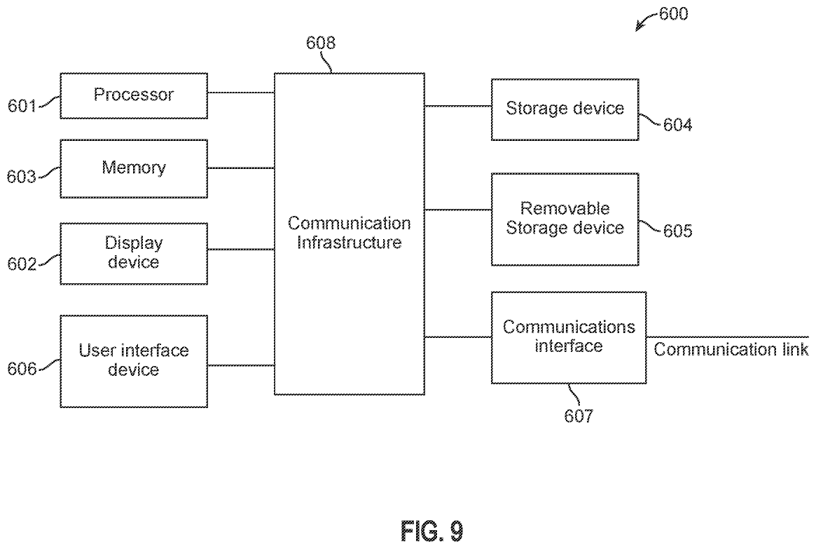

FIG. 9 is a high-level block diagram showing an information processing system comprising a computer system 600 useful for implementing the disclosed embodiments. One or more components of the telehealth system 400, the telehealth app 200, and/or the telehealth app 300 may be implemented using the computer system 600. The computer system 600 may be incorporated in a device 50 or a server device 110. The computer system 600 includes one or more processors 601, and can further include an electronic display device 602 (for displaying video, graphics, text, and other data), a main memory 603 (e.g., random access memory (RAM)), storage device 604 (e.g., hard disk drive), removable storage device 605 (e.g., removable storage drive, removable memory module, a magnetic tape drive, optical disk drive, computer readable medium having stored therein computer software and/or data), viewer interface device 606 (e.g., keyboard, touch screen, keypad, pointing device), and a communication interface 607 (e.g., modem, a network interface (such as an Ethernet card), a communications port, or a PCMCIA slot and card). The communication interface 607 allows software and data to be transferred between the computer system and external devices. The computer system 600 further includes a communications infrastructure 608 (e.g., a communications bus, cross-over bar, or network) to which the aforementioned devices/modules 601 through 607 are connected.

Information transferred via communications interface 607 may be in the form of signals such as electronic, electromagnetic, optical, or other signals capable of being received by communications interface 607, via a communication link that carries signals and may be implemented using wire or cable, fiber optics, a phone line, a cellular phone link, an radio frequency (RF) link, and/or other communication channels. Computer program instructions representing the block diagram and/or flowcharts herein may be loaded onto a computer, programmable data processing apparatus, or processing devices to cause a series of operations performed thereon to generate a computer implemented process. In one embodiment, processing instructions for process 800 (FIG. 8) may be stored as program instructions on the memory 603, storage device 604 and the removable storage device 605 for execution by the processor 601.

Embodiments have been described with reference to flowchart illustrations and/or block diagrams of methods, apparatus (systems) and computer program products. Each block of such illustrations/diagrams, or combinations thereof, can be implemented by computer program instructions. The computer program instructions when provided to a processor produce a machine, such that the instructions, which execute via the processor create means for implementing the functions/operations specified in the flowchart and/or block diagram. Each block in the flowchart/block diagrams may represent a hardware and/or software module or logic. In alternative implementations, the functions noted in the blocks may occur out of the order noted in the figures, concurrently, etc.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions, messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Computer program instructions may be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

As will be appreciated by one skilled in the art, aspects of the embodiments may be embodied as a system, method or computer program product. Accordingly, aspects of the embodiments may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the embodiments may take the form of a computer program product embodied in one or more computer readable medium(s) having computer readable program code embodied thereon.

Any combination of one or more computer readable medium(s) may be utilized. The computer readable medium may be a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Computer program code for carrying out operations for aspects of one or more embodiments may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of one or more embodiments are described above with reference to flowchart illustrations and/or block diagrams of methods, apparatus (systems) and computer program products. It will be understood that each block of the flowchart illustrations and/or block diagrams, and combinations of blocks in the flowchart illustrations and/or block diagrams, can be implemented by computer program instructions. These computer program instructions may be provided to a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods, and computer program products according to various embodiments. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

References in the claims to an element in the singular is not intended to mean “one and only” unless explicitly so stated, but rather “one or more.” All structural and functional equivalents to the elements of the above-described exemplary embodiment that are currently known or later come to be known to those of ordinary skill in the art are intended to be encompassed by the present claims. No claim element herein is to be construed under the provisions of 35 U.S.C. section 112, sixth paragraph, unless the element is expressly recited using the phrase “means for” or “step for.”

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the embodiments has been presented for purposes of illustration and description, but is not intended to be exhaustive or limited to the embodiments in the form disclosed. Many modifications and variations will be apparent to those of ordinary skill in the art without departing from the scope and spirit of the invention.

Though the embodiments have been described with reference to certain versions thereof; however, other versions are possible. Therefore, the spirit and scope of the appended claims should not be limited to the description of the preferred versions contained herein.

What is claimed is:

1. A method comprising:
providing, on a first device associated with a patient user, a questionnaire comprising questions inquiring about well-being of the patient user;
receiving, from the first device, responses of the patient user to the questionnaire;
generating a patient user profile by applying machine learning to the responses, wherein the patient user profile comprises diagnostic questions customized for the patient user;
providing, on a second device associated with a third party user, the patient user profile;
initiating, via the first device and the second device, a video teleconference between the patient user and the third party user, wherein the third party user asks the patient user the diagnostic questions during the video teleconference to assess and diagnose a mental health of the patient user; and
providing the patient user with a therapeutic treatment plan prescribed by the third party user based on the assessment and diagnosis, wherein the therapeutic treatment plan is indicative of at least one dosing session for the patient user to complete, frequency of the at least one dosing session, and an amount of psychedelics for the patient user to consume during the at least one dosing session.

2. The method of claim 1, wherein the third party user is a licensed therapist.

3. The method of claim 1, wherein each dosing session is one of a microdosing session or a high dose session.

4. The method of claim 1, further comprising:
requesting feedback from the patient user, wherein the feedback is indicative of an experience and effects of the patient user after a dosing session.

5. The method of claim 4, further comprising:
requesting updated responses of the patient user to the questionnaire at pre-determined intervals after a first dosing session.

6. The method of claim 5, further comprising:
monitoring satisfaction of the patient user with the therapeutic treatment plan based on at least one of the feedback or the updated responses.

7. The method of claim 6, further comprising:
requesting the third party user to make an adjustment to the therapeutic treatment plan based on at least one of the feedback, the updated responses, or the satisfaction of the patient user with the therapeutic treatment plan.

8. A system, comprising:
at least one processor; and
a non-transitory processor-readable memory device storing instructions that when executed by the at least one processor causes the at least one processor to perform operations including:
providing, on a first device associated with a patient user, a questionnaire comprising questions inquiring about well-being of the patient user;
receiving, from the first device, responses of the patient user to the questionnaire;
generating a patient user profile by applying machine learning to the responses, wherein the patient user profile comprises diagnostic questions customized for the patient user;
providing, on a second device associated with a third party user, the patient user profile;
initiating, via the first device and the second device, a video teleconference between the patient user and the third party user, wherein the third party user asks the patient user the diagnostic questions during the video teleconference to assess and diagnose a mental health of the patient user; and
providing the patient user with a therapeutic treatment plan prescribed by the third party user based on the assessment and diagnosis, wherein the therapeutic treatment plan is indicative of at least one dosing session for the patient user to complete, frequency of the at least one dosing session, and an amount of psychedelics for the patient user to consume during the at least one dosing session.

9. The system of claim 8, wherein the third party user is a licensed therapist.

10. The system of claim 8, wherein each dosing session is one of a microdosing session or a high dose session.

11. The system of claim 8, wherein the operations include:
requesting feedback from the patient user, wherein the feedback is indicative of an experience and effects of the patient user after a dosing session.

12. The system of claim 11, wherein the operations include:
requesting updated responses of the patient user to the questionnaire at pre-determined intervals after a first dosing session.

13. The system of claim 12, wherein the operations include:
monitoring satisfaction of the patient user with the therapeutic treatment plan based on at least one of the feedback or the updated responses.

14. The system of claim 13, wherein the operations include:
requesting the third party user to make an adjustment to the therapeutic treatment plan based on at least one of the feedback, the updated responses, or the satisfaction of the patient user with the therapeutic treatment plan.

15. A non-transitory processor-readable medium that includes a program that when executed by a processor performs a method comprising:
providing, on a first device associated with a patient user, a questionnaire comprising questions inquiring about well-being of the patient user;
receiving, from the first device, responses of the patient user to the questionnaire;
generating a patient user profile by applying machine learning to the responses, wherein the patient user profile comprises diagnostic questions customized for the patient user;
providing, on a second device associated with a third party user, the patient user profile;
initiating, via the first device and the second device, a video teleconference between the patient user and the third party user, wherein the third party user asks the patient user the diagnostic questions during the video teleconference to assess and diagnose a mental health of the patient user; and
providing the patient user with a therapeutic treatment plan prescribed by the third party user based on the assessment and diagnosis, wherein the therapeutic treatment plan is indicative of at least one dosing session for the patient user to complete, frequency of the at least one dosing session, and an amount of psychedelics for the patient user to consume during the at least one dosing session.

16. The non-transitory processor-readable medium of claim 15, wherein the third party user is a licensed therapist.

17. The non-transitory processor-readable medium of claim 15, wherein each dosing session is one of a microdosing session or a high dose session.

18. The non-transitory processor-readable medium of claim 15, wherein the method further comprises:
requesting feedback from the patient user, wherein the feedback is indicative of an experience and effects of the patient user after a dosing session.

19. The non-transitory processor-readable medium of claim 18, wherein the method further comprises:
requesting updated responses of the patient user to the questionnaire at pre-determined intervals after a first dosing session.

20. The non-transitory processor-readable medium of claim 19, wherein the method further comprises:
monitoring satisfaction of the patient user with the therapeutic treatment plan based on at least one of the feedback or the updated responses;
requesting the third party user to make an adjustment to the therapeutic treatment plan based on at least one of the feedback, the updated responses, or the satisfaction of the patient user with the therapeutic treatment plan; and
uploading and storing in one or more electronic medical records of the patient user the feedback, the updated responses, one or more certifications of the third party user, and at least one record of at least one session between the patient user and the third party user.
